(19) United States
(12) Patent Application Publication
(10) Pub. No.: US 2023/0383259 A1
(43) Pub. Date: Nov. 30, 2023

(54) IN VIVO ADIPOSE BIOREACTOR AND KITS FOR THE PRODUCTION AND DELIVERY OF BIOLOGIC AGENTS

(71) Applicant: The Brigham and Women's Hospital, Inc., Boston, MA (US)

(72) Inventor: Shailesh AGARWAL, Auburndale, MA (US)

(21) Appl. No.: 18/026,411

(22) PCT Filed: Sep. 15, 2021
(86) PCT No.: PCT/US2021/050424

§ 371 Date: Mar. 15, 2023

Related U.S. Application Data
(60) Provisional application No. 63/078,655, filed on Sep. 15, 2020.

(51) Int. Cl. C12N 5/077; C12N 9/22; C12N 15/86; C07K 14/495; C07K 14/71

(52) U.S. Cl. CPC C12N 5/0653; C12N 9/22; C12N 15/86; C07K 14/495; C07K 14/71; C12N 2510/00; C12N 2509/10; C12N 2310/20; C07K 2319/30; C12N 2740/15043

(57) ABSTRACT

Transgenic adipocytes that express and secrete a therapeutic biologic agent (TBA) are provided. Also provided are methods for producing these transgenic adipocytes and methods for using these transgenic adipocytes to treat a condition in a subject.

### CROSS-REFERENCE TO RELATED APPLICATIONS

This application priority to U.S. Provisional Application No. 63/078,655 filed on Sep. 15, 2020, the contents of which is incorporated by reference in its entirety.

### STATEMENT REGARDING FEDERALLY SPONSORED RESEARCH

N/A

### SEQUENCE LISTING

A Sequence Listing accompanies this application and is submitted as an ASCII text file of the sequence listing named “129319_00795_ST25.txt” which is 28,672 bytes in size and was created on Sep. 13, 2021. The sequence listing is electronically submitted via EFS-Web with the application and is incorporated herein by reference in its entirety.

### BACKGROUND

Therapeutic biologic agents (TBAs), in contrast to chemical or small molecule drugs produced through laboratory chemical synthesis, are therapeutic molecules derived from biological organisms. Classic TBAs include erythropoietin for anemia and insulin for diabetes. TBAs include therapeutic peptides (e.g., cytokines, antibodies, enzymes, fusion proteins) and nucleic acids (e.g., microRNAs), which have recently provided novel treatment options where none existed, such as α-galactosidase for Fabry disease and anti-TNFα for Crohn disease. The immense therapeutic potential of TBAs is supported by the fact that over 50 monoclonal antibodies have received regulatory approval, with over 300 currently in development.

Although there is growing demand for TBAs, manufacturing and delivery present substantial barriers to their clinical use. Manufacturing is a complex, multi-step process requiring cell culture, purification, and formulation. In vitro culture is performed with heterologous systems using mammalian cells, yeast, or E. coli, each with its own limitations. For example, E. coli are unable to perform post-translational glycosylation. Yeast engineered to produce humanized antibodies still exhibit non-human glycosylation, posing an immunogenicity risk. Chinese hamster ovary cells, a commonly used mammalian cell line, cannot perform the full range of human glycosylation and may add immunogenic modifications. Human in vitro systems employing human embryonic kidney (HEK293) cells may optimize human glycosylation patterns; however, their use is limited to transient peptide synthesis. In vitro production processes are also limited by expensive purification and formulation protocols, presenting a significant downstream bottleneck. These limitations lead to substantial financial cost and infrastructure requirements.

Production aside, TBA delivery presents another barrier. Administration is often performed via intermittent intravenous or self-administered subcutaneous injections due to bioavailability requirements. The former requires multiple visits to infusion centers, which present a barrier to access for patients. Repeat infusions can also lead to venous damage and chronic pain, as well as infusion reactions. Self-administered subcutaneous injections cause pain and bruising which diminish long-term adherence⁸. Although depot strategies are under investigation, these still are not feasible for a majority of TBAs due to biochemical properties which limit bioavailability. Scaffolds or hydrogels are often prone to inflammatory reactions which may damage local tissue, and the use of non-living implantable devices is often complicated by infection, extrusion, and tissue damage requiring surgical removal. Furthermore, scaffolds or hydrogels are able to provide drug for only a limited period of time until the reservoir is depleted. Thus, unlike a bioreactor, they are unable to supply the biologic agent for long-term delivery and current strategies to regulate or eliminate scaffolds are limited.

Accordingly, there remains a need in the art for improved methods for producing and delivering TBAs to target tissues treat disease.

### SUMMARY

In a first aspect, the present disclosure provides methods for treating a condition in a subject. The methods comprise: (a) obtaining adipose tissue from the subject; (b) dissociating the adipose tissue into individual cells; (c) isolating adipocytes from the individual cells generated in step (b); (d) transfecting the isolated adipocytes with a polynucleotide comprising a promoter operably linked to a transgene encoding a therapeutic biologic agent (TBA); and (e) introducing the transfected adipocytes into the subject. In these methods, the transfected adipocytes engraft into a target tissue and express and secrete the TBA, such that the TBA is available to treat the condition.

In a second aspect, the present disclosure provides methods for producing transfected adipocytes. The methods comprise: (a) obtaining a plurality of isolated adipocytes; and (b) transfecting, using electroporation, the plurality of adipocytes with a polynucleotide comprising a promoter operably linked to a transgene.

In a third aspect, the present disclosure provides isolated adipocytes transfected with a polynucleotide comprising a promoter operably linked to a transgene.

### BRIEF DESCRIPTION OF THE DRAWINGS

FIG. 1 is a flow chart depicting the disclosed strategy for using transgenic adipocytes as autograft bioreactors for therapeutic biologic agent (TBA) production and delivery.

FIG. 2 is a schematic depicting one embodiment of the disclosed strategy in which liposuction is used to remove adipose tissue from the patient and CRISPR/Cas9 gene editing is used to introduce a polynucleotide, encoding a TBA under the control of a doxycycline-inducible promoter, into the adipocyte genome.

FIG. 3 shows purification of adipocytes from human adipose tissue. (A) Processing of fat via mincing, homogenization, and filtration. (B) Purified adipocytes.

FIG. 4 shows survival of adipocytes after electroporation.

FIG. 5 shows expression of the transgenes Bmpr1αfc and Tgfbr2fc by transfected adipocytes.

FIG. 6 shows doxycycline-inducible expression of Bmpr1αfc and Tgfbr2fc in HEK293 cells.

FIG. 7 demonstrates that transfected adipocytes secrete BMPR1A-Fc and TGFβRII-Fc peptides.

FIG. 8 demonstrates that BMPR1A-Fc, TGFβRII-Fc, and insulin peptides secreted by transfected adipocytes exhibit bioactivity.

FIG. 9 shows engraftment of electroporated RFP+ mouse adipocytes in subcutaneous (A), intramuscular (B), and tendinous (C) tissues.

FIG. 10 demonstrates that treatment with poloxamer-188 following electroporation increased TBA expression levels in the transgenic adipocytes.

FIG. 11 demonstrates that treatment of adipocytes with poloxamer-188 results in downregulation of apoptosis-related genes in, including Bid and Caspase-3.

FIG. 12 demonstrates that conditioned media from HEK293 cells transfected with XMIR-LV-miR122 reduces reporter activity.

FIG. 13 shows components of systems for performing adipocyte processing (i.e., isolation and transfection).

### DETAILED DESCRIPTION

The present disclosure provides transgenic adipocytes that express and secrete a therapeutic biologic agent (TBA). Also provided are methods for producing these transgenic adipocytes and methods for using these transgenic adipocytes to treat a condition in a subject.

In vivo living bioreactors, i.e., transgenic cells that are programmed to produce a therapeutic biologic agent (TBA) of interest, offer an intriguing solution to many of the challenges associated with conventional therapies. First, they obviate financial and resource costs associated with in vitro cell culture, purification, and subsequent TBA formulation for delivery. Instead, the TBA is simply produced by the transgenic cells and eluted locally or systemically. With this strategy there is no need for repeated TBA administrations (i.e., via infusions or injections), which improves patient adherence as well as the ability to maintain therapeutic TBA levels over an extended period of time. Placement of in vivo bioreactors within deep structures would facilitate sustainable, local delivery in sites that are not amenable to repeated direct injections (e.g., internal organs, joints, deep muscles).

Recently, in vivo bioreactors composed of gene-edited bacteria (E. coli), cultured skin grafts, pancreatic islet cells, and multi-layered free flaps requiring microvascular surgery have been investigated for production and delivery of biologic agents. However, each of these strategies has significant barriers to clinical translation: (1) E. coli are not autologous and are unable to perform post-translational glycosylation, which is required for human biologic agents, (2) cultured skin grafts are costly to produce, require painful and unsightly recipient-site scars, cannot be placed within muscle or joints, are prone to breakdown and wounds due to the absence of a durable extracellular matrix, and are at risk for malignant degeneration, (3) pancreatic islet cell procurement requires an intra-abdominal procedure and yields only a small number of cells which must undergo in vitro expansion, and (4) microvascular free flaps are technically demanding and are not amenable to electroporation due to their large, solid structure and because delivery of an electric current would cause endothelial disruption, intra-flap thrombosis, and flap loss.

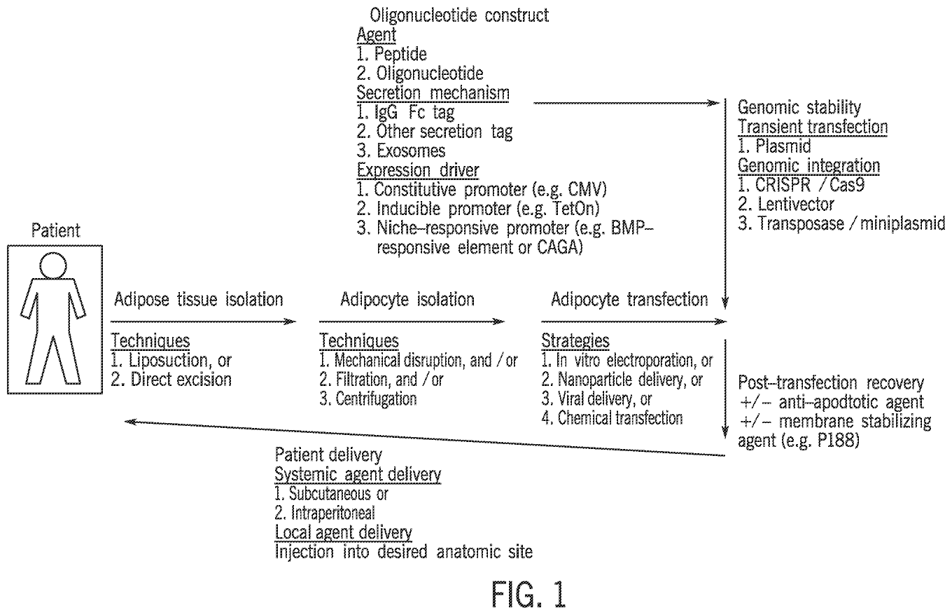

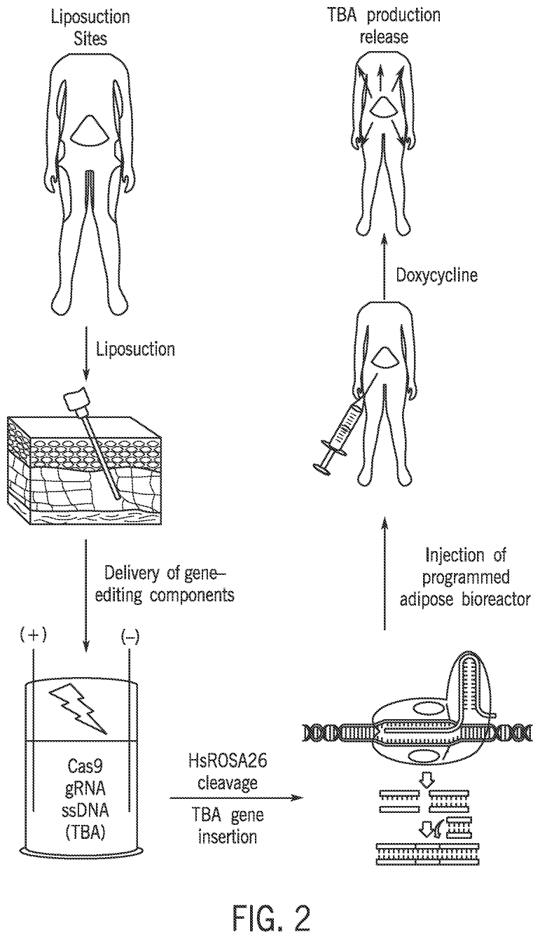

To overcome these limitations, the present investigators have developed a strategy in which autologous adipocytes serve as long-term, endogenous bioreactors for the expression and secretion of TBAs. In this strategy, adipose tissue is harvested from a subject and mechanically separated and purified into adipocytes, which are then transfected with a polynucleotide encoding a TBA and reintroduced into the subject as an autologous graft (FIG. 1). For example, in one particular embodiment, liposuction is used to remove adipose tissue from a human subject in the operating room, individual adipocytes are isolated from the adipose tissue, CRISPR/Cas9-based gene editing is used to introduce a doxycycline-inducible transgene encoding the TBA into the adipocyte genome, and the transgenic adipocytes are reinjected into the subject (FIG. 2).

The disclosed TBA delivery strategy offers at least three significant advantages. First, human tissue makes a desirable bioreactor for human therapies as it provides the cellular machinery required for post-translational modification, reducing the risk of an immunologic response. Human tissue can be obtained from the patient requiring treatment to be used an autologous graft, further reducing or eliminating the risk of rejection. Adoption of gene editing techniques, such as CRISPR and lentivector- or transposase-mediated genomic recombination, has made it possible to insert whole transgenes into the genome of human tissue.

Second, the adipocytes used in the disclosed strategy are derived from adipose tissue, which is a desirable tissue for the creation of autologous in vivo bioreactors because it is abundant, has low cellular turnover and high cellularity, and is easy to harvest. The high abundance of adipocytes within the adipose tissue of patients avoids the need for in vitro cell expansion, allowing the method to be accomplished using a patient's own cells in the operating room. Further, adipose tissue is easily accessible with minimal donor site morbidity, and removal is routinely performed by surgeons. For example, adipose tissue may be isolated in a semi-solid state from consenting human patients using liposuction in the operating room as part of routine reconstructive or aesthetic surgery. This tissue would otherwise be discarded as excess. A typical liposuction cannula inserted through a small (4-5 mm) skin incision harvests tissue volumes up to 4-5 liters, of which adipocytes make up approximately 83% by volume. Unlike skin graft donor sites, liposuction sites exhibit minimal morbidity and no scarring. Likewise, the recipient sites experience no morbidity, as the adipocyte graft can be delivered through minimally invasive needles. Prior to the work presented in this application, no other team has reported successful gene delivery to mature human primary adipocytes. Previous work has been performed with transfected mesenchymal cells or pre-adipocytes but these are not amenable to human therapeutics, as these cells are not present abundantly and, therefore, require in vitro expansion. Further, unlike adipocytes, mesenchymal cells are difficult to isolate from the adipose tissue because they are located within the stroma and do not easily dissociate.

Third, the use of ex vivo electroporation for gene delivery followed by immediate reintroduction of the transfected cells back into the donor avoids the need for subsequent in vitro culture and provides the advantages outlined in Table 1. Further, this strategy (1) reduces the need for tissue handling, (2) eliminates the infrastructure and resource requirements of in vitro cell expansion, and (3) reduces the risk for mutation accrual or malignant transformation during in vitro cell expansion.

TABLE 1
Advantages of gene delivery via ex vivo electroporation

| | Ex vivo | In vivo | Ex vivo virus | In vivo virus |
|---|---|---|---|---|
| Advantage | electroporation | electroporation | or nanoparticle | or nanoparticle |
| High-efficiency tissue delivery | Yes | Yes | No | No |
| Delivery only to targeted | Yes | No | Yes | No |
| cells/tissues, enabling precise | | | | |
| excision of transfected tissues | | | | |
| if desired | | | | |
| Minimal/no immune response | Yes | Yes | No | No |
| Minimal/no incubation time | Yes | Yes | No | No |
| Safe and feasible delivery to | Yes, aliquots | No | Yes | Yes |
| large tissue volumes | | | | |
| Delivery of recombinant | Yes | Yes | Yes, nanoparticle | Yes, nanoparticle |
| protein (e.g. recombinant Cas9) | | | | |

### 1—Methods for Treating a Condition in a Subject:

In a first aspect, the present disclosure provides methods for treating a condition in a subject. The methods comprise: (a) obtaining adipose tissue from the subject; (b) dissociating the adipose tissue into individual cells; (c) isolating adipocytes from the individual cells generated in step (b); (d) transfecting the isolated adipocytes with a polynucleotide comprising a promoter operably linked to a transgene encoding a therapeutic biologic agent (TBA); and (e) introducing the transfected adipocytes into the subject. In these methods, the transfected adipocytes engraft into a target tissue and express and secrete the TBA, such that the TBA is available to treat the condition.

In step (a) of the methods, adipose tissue is obtained from the subject. “Adipose tissue”, which is also referred to as “fat” or “body fat”, is a loose connective tissue composed of adipocytes, preadipocytes, fibroblasts, vascular endothelial cells, and a variety of immune cells (e.g., adipose tissue macrophages). Adipose tissue acts as an energy depot and helps to cushion and insulate the body.

Adipose tissue may be harvested in a semi-solid state from the subject using liposuction or may be harvested as whole adipose tissue using surgical techniques. Suitable surgical techniques include, for example, direct subcutaneous excision, as is performed in abdominoplasty. Thus, in some embodiments, the adipose tissue is obtained as a liquified liposuction aspirate or as whole adipose tissue. In embodiments in which whole adipose tissue is harvested, it may be advantageous to separate the adipose tissue into pieces (e.g., 1-2 mm pieces) to ensure that dissociation can be performed efficiently. Adipose tissue may be broken into smaller pieces using mechanical methods such as chopping, mincing, grinding, and the like.

In step (b) of the methods, adipose tissue is dissociated into individual cells. Dissociation produces a cell suspension that is amenable to electroporation, unlike whole adipose tissue. Tissue dissociation can be accomplished using enzymatic dissociation, chemical dissociation or mechanical dissociation. In enzymatic dissociation, enzymes are used to digest the tissue into individual cells. Suitable enzymes for use in enzymatic dissociation of adipose tissue include, without limitation, collagenase, trypsin, dispase, and combinations thereof. In chemical dissociation, EDTA or EGTA are used to bind cations, thereby disrupting intercellular bonds. In mechanical dissociation, the tissue is cut, scraped, or scratched into small pieces. This may be accomplished using a device such as a tissue dissociator. For instance, a gentleMACS™ Dissociator was used to dissociate adipose tissue in the Examples. The minced-up tissue produced by mechanical dissociation may then be washed to separate the cells from the tissue. Sometimes gentle agitation is also used to help loosen the cells. While any of the above methods can be used to dissociate the adipose tissue, mechanical dissociation offers several advantages over the other methods, including that it (1) avoids long incubation periods, making it amenable to point-of-care use; and (2) reduces the number of exogenous agents that are introduced into the subject, thereby lowering the bar for Food and Drug Administration approval. Thus, in some embodiments, the dissociation of the adipose tissue into individual cells is accomplished using mechanical dissociation.

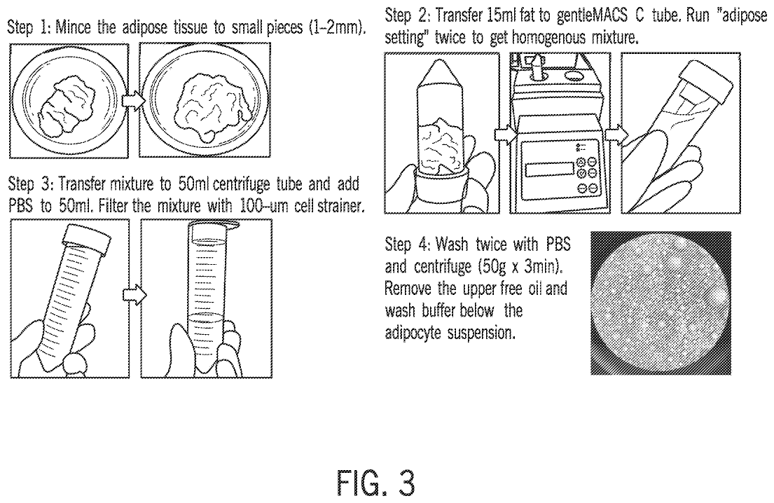

In step (c) of the methods, adipocytes are isolated from the individual cells generated in step (b). The use of isolated adipocytes rather than adipose tissue produces a more homogenous therapeutic cell type, which improves the likelihood of successful gene delivery using a single set of conditions. As is described in Example 1, adipocytes were isolated from by filtering the cells through a strainer, centrifuging the filtrate at 50 g for 3 minutes to separate the mixture into a top layer comprising free oil, a middle layer comprising dissociated adipocytes, and a bottom layer comprising buffer, and removing the top layer and bottom layer to isolate the middle layer comprising the adipocytes (see FIG. 3). Thus, in some embodiments, isolating adipocytes comprises (i) filtering the individual cells generated in step (b) to obtain a filtrate; (ii) centrifuging the filtrate; and (iii) harvesting the adipocytes from the centrifuged filtrate.

A filter is used with the present invention to separate cells (which pass through the filter into the filtrate) from stroma (which is retained in the oversize). Adipocytes can be up to 300 μm in diameter. Thus, the filter used with the present invention may have a pore size of about 50 μm to about 300 μm. In the Examples, the inventors utilized a filter with a 100 μm pore size. Thus, in some embodiments, the filter has a pore size of 100 μm.

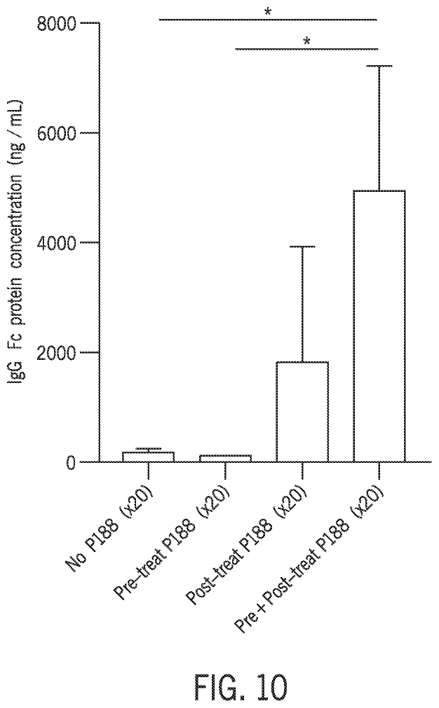

In some embodiments, the methods further comprise treating the adipocytes with a drug that improves cell survival. Suitable drugs that improve cell survival include, without limitation, anti-apoptotic agents, BCL-2 inhibitors, and membrane stabilizing agents. For example, treatment of adipocytes with the polymeric compound poloxamer-188 is known to stabilize the cell membrane after cellular injury. In Example 1, it is demonstrated that treatment of the adipocytes with poloxamer-188 after electroporation resulting in increased transgene expression levels, suggesting that the treatment improved adipocyte stability (see FIG. 10). However, treatment with poloxamer-188 prior to electroporation appears to reduce transfection efficiency. Thus, in some embodiments, the methods further comprise treating the transfected adipocytes with poloxamer-188 following electroporation.

In step (d) of the methods, the isolated adipocytes are transfected with a polynucleotide comprising a promoter operably linked to a transgene encoding a therapeutic biologic agent (TBA). As used herein, the term “transfection” is used to describe a process in which a nucleic acid is deliberately introduced into a cell. Likewise, a “transfected cell” is a cell into which nucleic acids have been deliberately introduced.

The term “polynucleotide” refers a polymer of DNA or RNA. A polynucleotide may be single-stranded or double-stranded, synthesized, or obtained (e.g., isolated and/or purified) from a natural source. A polynucleotide may contain natural, non-natural, or altered nucleotides, as well as natural, non-natural, or altered internucleotide linkages (e.g., a phosphoroamidate linkage or a phosphorothioate linkage). The term polynucleotide encompasses constructs (i.e., artificially constructed DNA molecules), plasmids, vectors, and the like.

As used herein, the term “promoter” refers to a DNA sequence that regulates the expression of a gene. This term is commonly used to refer to a regulatory region that is capable of recruiting RNA polymerase and initiating transcription of a downstream (3′ direction) sequence. However, as used herein, this term encompasses enhancers and other transcriptional regulatory elements. A promoter may be located at the 5′ or 3′ end, within a coding region, or within an intron of a gene that it regulates. Promoters may be derived in their entirety from a native gene, may be composed of elements derived from multiple regulatory sequences found in nature, or may comprise synthetic DNA segments. It is understood by those skilled in the art that different promoters may direct the expression of a gene in different tissues or cell types, at different stages of development, or in response to different environmental conditions. A promoter is “operably linked” to a polynucleotide if the promoter is connected to the polynucleotide such that it may affect transcription of the polynucleotide.

In some embodiments, the promoter used in the present disclosure is a constitutive promoter, inducible promoter, or niche-responsive promoter. In some embodiments, the promoter is a “constitutive promoter”, i.e., a promoter that causes a gene to be expressed in most cell types at most times. In the examples, the constitutive cytomegalovirus (CMV) immediate early promoter has been used to drive transgene expression. Thus, in some embodiments, the promoter is a CMV promoter. Other suitable constitutive promoters include, without limitation, the elongation factor-1 alpha (EF-1 alpha) promoter and the U6 promoter, which is a type III RNA polymerase III promoter that is commonly used to drive expression of small RNAs. In other embodiments, the promoter is an “inducible promoter”, i.e., a promoter that allows for controlled expression of a gene. A practitioner may activate (i.e., induce) an inducible promoter by subjecting the promoter to a particular condition (e.g., heat) or to the presence of a particular molecule (e.g., doxycycline). In the examples, a Tet-On system was used to regulate TBA expression. In this system, the protein reverse tetracycline transactivator (rtTA) is only able to bind to a tetracycline response element (TRE) and initiate transcription after it has bound to doxycycline. Thus, the Tet-On system is said to be doxycycline-inducible. In some embodiments, the promoter is a “niche-responsive promoter”, i.e., a promoter that is activated in response to a signal present in the cellular environment. For example, a niche-responsive promoter may be activated by a stimulus (e.g., light or a mechanical force) or an agent such as a specific ligand (e.g., glucose, TGFB, BMP). For example, a niche-responsive promoter that is upregulated during hyperglycemia may be used to drive insulin expression for the treatment of type I diabetes. Suitable niche responsive promoters include, without limitation, the Col1 promoter (which is activated in the presence of TGFB1) and promoters that comprise a BMP-responsive element or a CAGA regulatory motif (e.g., a CAGA box).

As used herein, the term “transgene” refers to a nucleotide sequence that is artificially (i.e., through human intervention) introduced into a cell. A transgene may comprise a naturally occurring gene, a synthetic gene, or a combination thereof. Likewise, a “transgenic cell” is a cell into which a transgene has been introduced.

The transgenic adipocytes of the present disclosure include a transgene encoding a therapeutic biologic agent. As used herein, the term “therapeutic biologic agent (TBA)” refers to a gene product (e.g., a protein or RNA) that can be used to treat a condition. Suitable TBAs include, but are not limited to, hormones, growth factors, antibodies, cytokines, receptor-IgG fusion proteins, microRNAs (miRNAs), small interfering RNAs (siRNAs), and short hairpin RNA (shRNAs). Specific examples of TBAs include (1) IL-12, a cytokine that may be used to treat malignancies; (2) anti-TNFα antibodies, which are used to treat autoimmune conditions (e.g., Crohn's disease or rheumatoid arthritis); (3) PDGF-B, a growth factor that is applied topically to improve wound healing; (4) VEGF, a growth factor that stimulates wound healing; (5) BMPR1A-Fc, a BMP receptor-IgG fusion protein that inhibits BMP signaling and can be used to treat heterotopic ossification; (6) TGFβRII-Fc, a transforming growth factor-β (TGFβ) receptor-IgG fusion protein that inhibits TGFβ ligands and can be used to treat heterotopic ossification or fibrosis; (7) insulin, a hormone that is used to treat type I diabetes; and (8) miR-497, a microRNA that functions as a tumor suppressor gene. In Example 1, it is demonstrated (1) that adipocytes transfected with polynucleotides encoding the TBAs TGFβRII-Fc, BMPR1A-Fc, and insulin secrete these TBAs, and (2) that the secreted TBAs are bioactive. Thus, in some embodiments, the TBA is BMPR1A-Fc, TGFβRII-Fc, or insulin.

BMPR1A-Fc is a fusion protein comprising the native signal peptide of the bone morphogenetic protein receptor type-1A (BMPR1A) protein, the functional domain of the BMPR1A) protein, a linker peptide, and an IgG-Fc tag. The amino acid sequence of this fusion protein is provided as SEQ ID NO:1 and the DNA sequence encoding this fusion protein is provided as SEQ ID NO:2. The amino acid sequence of BMPR1A, from which the signal peptide and functional domain are derived, is provided as SEQ ID NO:5. The BMPR1A signal peptide is provided as amino acids 1-23 of SEQ ID NO:1 and amino acids 1-23 of SEQ ID NO:5. The BMPR1A functional domain is provided as amino acids 24-152 of SEQ ID NO:1 and amino acids 24-152 of SEQ ID NO:5. The linker peptide used in this fusion protein is provided as SEQ ID NO:7. The IgG-Fc tag is a c-terminal portion of the fragment crystallizable region (Fc region) of immunoglobulin heavy constant gamma 1. The amino acid sequence of the full-length Fc region is provided as SEQ ID NO:8, and the portion of this protein that used as an IgG-Fc tag (i.e., amino acids 100-330 of SEQ ID NO:8) is provided as SEQ ID NO:9. BMPR1A-Fc is commercially available from several companies.

Likewise, TGFβRII-Fc is a fusion protein comprising the native signal peptide of the TGF-beta receptor type-2 isoform B (TGFβRII) protein, the functional domain of the TGFβRII protein, a linker peptide, and an IgG-Fc tag. The amino acid sequence of this fusion protein is provided as SEQ ID NO:3 and the DNA sequence encoding this fusion protein is provided as SEQ ID NO:4. The amino acid sequence of TGFβRII, from which the signal peptide and functional domain are derived, is provided as SEQ ID NO:6. The TGFβRII signal peptide is provided as amino acids 1-22 of SEQ ID NO:3 and amino acids 1-22 of SEQ ID NO:6. The TGFβRII functional domain is provided as amino acids 23-159 of SEQ ID NO:1 and amino acids 23-159 of SEQ ID NO:5. The linker peptide used in this fusion protein comprises a methionine followed by an aspartic acid (i.e., MD). As in BMPR1A-Fc, the IgG-Fc tag is a c-terminal portion of the fragment crystallizable region (Fc region) of immunoglobulin heavy constant gamma 1. The amino acid sequence of the full-length Fc region is provided as SEQ ID NO:8, and the portion of this protein that used as an IgG-Fc tag (i.e., amino acids 100-330 of SEQ ID NO:8) is provided as SEQ ID NO:9. TGFβRII is commercially available from several companies.

In the present methods, the transfected adipocytes must secrete the TBA following engraftment into the target tissue. TBA secretion can be accomplished using several mechanisms. In some embodiments, the TBA comprises a native signal peptide that is sufficient for secretion (e.g., the native signal peptide of TGFβRII or BMPR1A). However, in other embodiments, an exogenous secretion tag may be added to the TBA to ensure that it is secreted. In other embodiments, the secretion tag is a tag that targets the TBA for exosome packaging. In Example 2, targeted microRNAs for exosome packaging using a specific RNA sequence tag provided in the XMIR Lentivector from System Biosciences. The use of exosomes to package TBAs offers several advantages: (1) exosome packaging may allow for increased long-term bioavailability of the TBAs, and (2) exosomes can be labeled with tags that specifically target them to a target cell (e.g., a tumor cell). Thus, in some embodiments, the TBA is packaged into an exosome within the transfected adipocytes.

In some embodiments, the TBA further comprises a solubility tag, i.e., a peptide tag that increases the solubility of a protein to which it is added. In Example 1, an IgG-Fc tag, which comprises the constant region of an immunoglobulin heavy-chain, is added to the TBAs TGFβRII and BMPR1A. The addition of this tag is known to increase protein solubility and expression yield. Thus, in some embodiments, the solubility tag is an IgG-Fc tag.

In certain embodiments, the transgene may further include a so-called “kill switch” to facilitate killing of the transfected cells, for example after they have been delivered to the subject, by administering a drug or other compound to the subject. The kill switch may be implemented by incorporation of a suicide gene (e.g., HSV-tk) into the transgene which induces cell death upon exposure to an administered compound, e.g., ganciclovir may be used when the suicide gene is HSV-tk. Including a kill switch allows for elimination of implanted adipocytes, for example if it is determined that the implanted adipocytes are not functioning as intended or that the intended treatment is completed or otherwise no longer wanted or needed.

Transfection may be accomplished using any suitable method known in the art including, without limitation, chemical transfection methods (e.g., using calcium phosphate), mechanical transfection methods (e.g., via electroporation, sonoporation, biolistic delivery, magnetic nanoparticles), and viral transfection methods (e.g., using adeno-associated virus). While nanoparticle-based or viral transfection methods may offer improved cell survival, these methods may cause the adipocytes to initiate an innate immune response, require a longer incubation time that electroporation, and may be less efficient than electroporation. Thus, in preferred embodiments, transfection is accomplished using electroporation.

Any method of gene delivery may be used to introduce the transgene into the adipocytes. Suitable methods include, without limitation, transient transfection using a circular plasmid and random genomic integration using a lentivirus, linearized plasmid, or transposase system (e.g., Sleeping Beauty). Thus, in some embodiments, the polynucleotide is a plasmid, miniplasmid, or lentiviral vector. However, targeted genomic integration via gene editing may be preferable because it allows for the generation of a reproducible, genomically stable in vivo bioreactor. “Gene editing” is a form of genetic engineering in which DNA is inserted, deleted, modified, or replaced at a specific site within the genome. Suitable gene editing methods include methods that that utilize gene editing enzymes such as meganucleases, zinc finger nucleases (ZFN), transcription activator-like effector nucleases (TALENs), and Cas nucleases.

Advantageously, CRISPR technology allows for site-directed integration (e.g., into recognized safe-harbor genomic sites), minimizing the potential for genetic disruption or random genomic integration. Thus, in preferred embodiments, gene editing is performed using the CRISPR/Cas9 system. In this system, Cas9 is recruited to a target genomic locus using a guide nucleic acid that is homologous to the target locus. Cas9 creates a double-stranded at the target locus, and at some frequency the cell will repair that the break using a provided “DNA donor template”, i.e., DNA that comprises the transgene construct flanked by sequences at the 5′ and 3′ends that are homologous to genomic sequences surrounding the target locus (i.e., “homology arms”). Thus, in some embodiments, the polynucleotide encoding the TBA is supplied as a DNA donor template and CRISPR is used to integrate the polynucleotide into a specific target locus within the adipocyte genome. In these embodiments, the methods further comprise transfecting the adipocytes with a gene editing enzyme (e.g., Cas9) and one or more guide nucleic acids (e.g., single guide RNAs). The gene editing enzyme may be delivered to the adipocytes as either a protein or as a nucleic acid construct encoding the enzyme. The DNA donor template may be supplied as either a single-stranded DNA (ssDNA) or a plasmid comprising the desired nucleotide sequence flanked by homology arms.

To achieve reproducible gene editing it may be advantageous to insert the polynucleotide into a safe harbor site within the adipocyte genome. A “safe harbor site” is a genomic site that is able to accommodate new genetic material in a manner that ensures that the newly inserted genetic elements: (i) function predictably and (ii) do not cause alterations of the host genome, which could pose a risk to the host cell. Thus, in some embodiments, the polynucleotide is a donor template that comprises homology arms that target a safe harbor site within the adipocyte genome.

In step (e) of the methods, the transfected adipocytes are introduced into the subject. In some embodiments, the adipocytes are introduced in a manner that allows for systemic TBA delivery, e.g., by subcutaneous injection or intraperitoneal injection. In some embodiments, the adipocytes are introduced in a manner that allows for local TBA delivery, e.g., by injection into a specific anatomic site. In some cases, introduction is accomplished using minimally invasive needles. In some cases (e.g., when the target tissue is a difficult to access internal tissue), surgery may be used to expose the target tissue to facilitate deposition of the transfected adipocytes into the correct tissue.

The “subject” to which the methods are applied may be a mammal or a non-mammalian animal, such as a bird. Suitable mammals include, but are not limited to, humans, cows, horses, sheep, pigs, goats, rabbits, dogs, cats, bats, mice and rats. In certain embodiments, the methods may be performed on lab animals (e.g., mice, rats, pigs) for research purposes. In other embodiments, the methods are used to treat commercially important farm animals (e.g., cows, horses, pigs, rabbits, goats, sheep, and chickens) or companion animals (e.g., cats and dogs). In a preferred embodiment, the subject is a human.

After the transfected adipocytes are introduced into the subject, they engraft into a target tissue and express and secrete the TBA. Expression and secretion of a protein TBA can be detected using any method of protein detection including, without limitation, enzyme-linked immunosorbent assay (ELISA), flow cytometry, western blotting, chromatographic methods, and protein mass spectrometry. Antibodies that bind to specific proteins are well-known in the art and some are commercially available, as are ELISA kits. Expression and secretion of a RNA TBA can be detected using any method of RNA detection including, without limitation, reverse transcription and polymerase chain reaction, (RT-PCR), Northern blotting, microarray analysis, and RNA sequencing. To facilitate detection, the RNA may first be isolated from cells using an RNA extraction technique, such as guanidinium thiocyanate-phenol-chloroform extraction (e.g., using TRIzol), trichloroacetic acid/acetone precipitation followed by phenol extraction, or using a commercially available column-based system (e.g., RNeasy RNA Preparation Kit from Qiagen). Such techniques are well known in the art.

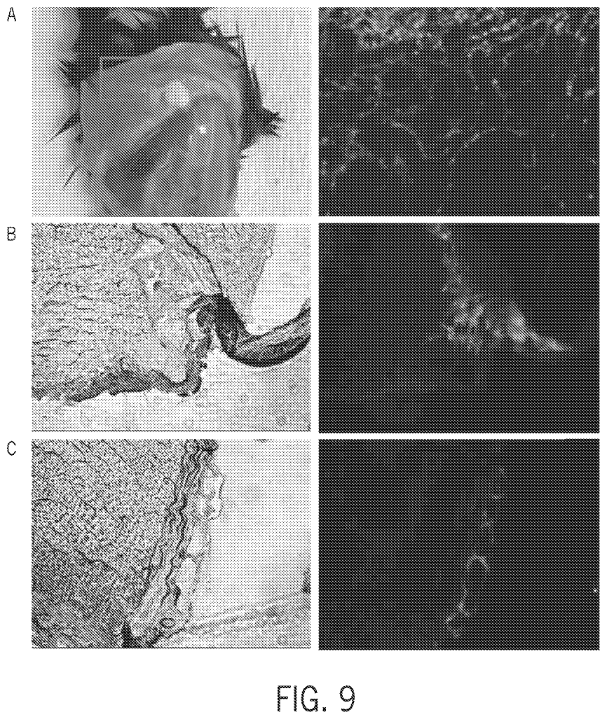

A cell is considered to have “engrafted” into a target tissue if it remains viable and functional following its introduction. The “target tissue” may be any type of tissue that would benefit from treatment with the TBA. Exemplary target tissues include muscle, bone, skin, nerves, kidney, liver, heart, brain, and other organs. In Example 1, the inventors demonstrated that the transfected adipocytes successfully engraft (i.e., survive and express a fluorescent protein) into subcutaneous dorsum, gastrocnemius muscle, or Achilles tendon of mice (see FIG. 9). Thus, in some embodiments, the target tissue is a tissue selected from the group consisting of subcutaneous tissue, muscle, and tendon.

Any condition that can be treated using a TBA may be treated using the present methods. As used herein, the term “condition” refers to a state of health. This term encompasses diseases, lesions, disorders, mental illnesses, nonpathologic conditions (e.g., pregnancy), and predisposing conditions (e.g., obesity). Exemplary conditions that may be treated using the present methods include, without limitation, autoimmune disorders, metabolic conditions, malignancies, and pathologic wounds. For example, in one specific embodiment, the condition is heterotopic ossification and the TBA is BMPR1A-Fc or TGFβRII-Fc. In a second specific embodiment, the condition is muscle fibrosis and the TBA is TGFβRII-Fc. In a third specific embodiments, the condition is type I diabetes and the TBA is insulin. In a fourth specific embodiment, the condition is lymphedema and the TBA is VEGF-C or TGFβRII-Fc.

### 2—Methods for Producing Transfected Adipocytes:

In a second aspect, the present disclosure provides methods for producing transfected adipocytes. The methods comprise: (a) obtaining a plurality of isolated adipocytes; and (b) transfecting, using electroporation, the plurality of adipocytes with a polynucleotide comprising a promoter operably linked to a transgene.

In some embodiments of the methods, obtaining the plurality of adipocytes comprises dissociating adipose tissue into individual cells and isolating the dissociated adipocytes. Suitable methods for dissociating adipose tissue into individual cells and methods for isolating the dissociated adipocytes are described in section 1 above (see the description of step (b) and step (c), respectively).

“Electroporation” is a physical transfection method that uses an electrical pulse to create temporary pores in cell membranes through which substances like nucleic acids can pass into cells. In the examples, experiments are described in which a variety of electroporation settings for transfection of adipocytes are tested, e.g., 100-1000V, 1-10 pulses, and 1-10 msec. Based on these experiments, it has been determined that the optimal electroporation settings for gene delivery and adipocyte survival are 500V, 4 pulses, and 5 msec per pulse. Thus, in some embodiments, electroporation is performed using those optimized electroporation settings.

As is described in section 1, treating adipocytes with poloxamer-188 post-electroporation appears to improve adipocyte stability. Thus, in some embodiments, the methods further comprise treating the transfected adipocytes with poloxamer-188 following electroporation.

In some embodiments, the transgene that is transfected into the adipocytes encodes a TBA, and the transfected adipocytes express and secrete the TBA. Exemplary TBAs for use with embodiments of the present disclosure are described in section 1 above. In some embodiments, the TBA is BMPR1A-Fc, TGFβRII-Fc, or insulin. In some embodiments, the TBA is packaged into an exosome within the transfected adipocytes.

As is described in section 1, any method of gene delivery may be used to introduce the transgene into the adipocytes. Thus, in some embodiments, the polynucleotide is a plasmid or lentiviral vector. In other embodiments, the polynucleotide is a DNA donor template and the methods further comprise transfecting the adipocytes with a gene editing enzyme and one or more guide nucleic acids. In specific embodiments, the gene editing enzyme is Cas9 and/or the DNA donor template comprises homology arms that target a safe harbor site within the adipocyte genome.

As is described in section 1, any promoter may be included in the polynucleotide to drive expression of the transgene. In some embodiments, the promoter is a constitutive promoter (e.g., the cytomegalovirus (CMV) immediate early promoter). In other embodiments, the promoter is an inducible promoter (e.g., part of a Tet-On system). In other embodiments, the promoter is a niche-responsive promoter.

The present disclosure further encompasses adipocytes produced using the any of the methods for producing transfected adipocytes described herein.

### 3—Transfected Adipocytes:

In a third aspect, the present disclosure provides isolated adipocytes transfected with a polynucleotide comprising a promoter operably linked to a transgene.

As used herein, the term “isolated” refers to a material that is substantially or essentially free from components that normally accompany it in its native state. Thus, an isolated adipocyte is an adipocyte that been purified away from the other cell types naturally found in adipose tissue.

Prior to the work of the present disclosure, successful gene delivery had not been achieved in primary adipocytes. Thus, in some embodiments, the adipocytes are mature, primary adipocytes. In some embodiments, the adipocytes are human adipocytes.

In some embodiments, the adipocytes comprise a transgene that encodes a TBA, and the adipocytes express and secrete the TBA. Exemplary TBAs for use with the present disclosure are described in section 1, above. In some embodiments, the TBA is BMPR1A-Fc, TGFβRII-Fc, or insulin. In some embodiments, the TBA is packaged into an exosome within the adipocytes.

As is described in section 1, any method of gene delivery may be used to introduce the transgene into the adipocytes. However, in preferred embodiments, the polynucleotide is stably integrated into the genome of the adipocyte. In specific embodiments, the polynucleotide is integrated into a safe harbor site within the adipocyte genome.

As is described in section 1, any promoter may be included in the polynucleotide to drive expression of the transgene. In some embodiments, the promoter is a constitutive promoter (e.g., the cytomegalovirus (CMV) immediate early promoter). In other embodiments, the promoter is an inducible promoter (e.g., part of a Tet-On system). In other embodiments, the promoter is a niche-responsive promoter.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the disclosure or of the appended claims.

### EXAMPLES

### Example 1

In the following example, the development of an autologous cell therapy is described which uses gene-edited adipocytes as bioreactors that express a desired transgene in a target tissue. Their therapy is designed to enable point-of-care delivery in an operating room environment.

In this example, a therapeutic strategy is validated using human or mouse adipocytes programmed to express transgenes encoding an array of therapeutic biologic agents (TBAs) such as peptides. Further, it is demonstrated that adipocytes comprising these transgenes are able to engraft in target tissues, including the subcutaneous tissue, muscle, and tendon.

1—Human Adipocytes can be Mechanically Isolated from Adipose Tissue

We have obtained fat specimens from mice or from human patients undergoing reconstructive or cosmetic plastic surgery procedures. We have developed and implemented a strategy for mechanical isolation of adipocytes from human (and mouse) fat tissue to obtain a purified population of adipocytes. The isolation of a purified cell population is critical to ensuring a homogenous therapy with reproducible expression and kinetic profiles. An approach for adipocyte isolation from human tissues has not been previously implemented or described due to concerns regarding adipocyte fragility. Our approach is innovative because it does not require enzymatic digestion, thereby obviating time-consuming incubation periods which are not amenable to the operating room and eliminating harsh conditions that would threaten adipocyte viability. As a result, our entire isolation protocol requires only 15 minutes and can be completed in a resource-restricted environment, such as the operating room. The procedure requires separation of whole adipose tissue into globules ranging in size from 1-10 cc or use of liquified liposuction aspirate, followed by mechanical dissociation using the gentleMACS Dissociator, filtration using a 100 μm cell strainer (which captures adipocytes while allowing smaller cell types to pass through), centrifugation (50 g×3 mins), and harvest of floating adipocytes (FIG. 3). We consistently obtain purified adipocytes using this approach.

### 2—Electroporation-Mediated Gene Delivery Enables Human Adipocytes to Express Transgenes Encoding Therapeutic Peptides

We have developed a point-of-care strategy for gene delivery into human adipocytes. Prior to our experiments, no other studies have demonstrated effective gene delivery into mature human adipocytes. While some studies have reported on gene delivery into 3T3-L1 adipocytes, our approach is transformative because we perform gene delivery into a primary adipocyte cell population. 3T3-L1 adipocytes are an adipocyte population which have been derived in vitro and are not primary cell population.

To accomplish our desired goal for point-of-care gene delivery, we have focused on electroporation-mediated delivery. Use of electroporation as a gene delivery strategy is superior to alternative strategies, such as virus-, nanoparticle- or chemical-based transfection, which require lengthy incubation periods. Furthermore, virus-based transfection risks virus introduction into the patient when the adipocytes are re-injected. Alternative vectors, such as nanoparticles, present a risk of off-target cell transfection.

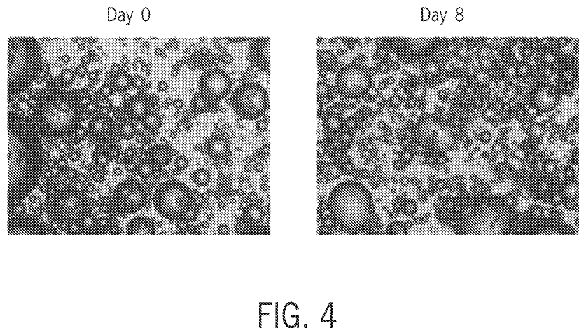

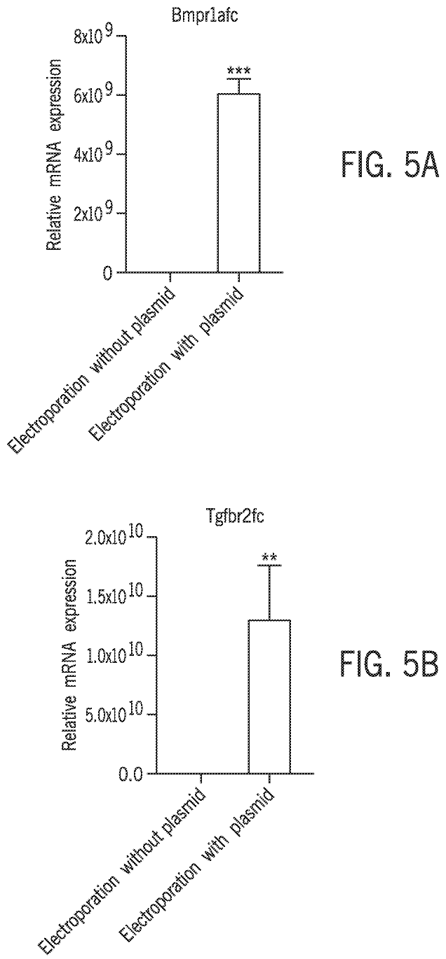

We tested a range of voltages (V), pulse number (#), and pulse width (msec), and have identified optimal gene-delivery conditions based on adipocyte survival (FIG. 4). We use the electroporation settings 500V/4 pulses/5 msec per pulse to deliver 20 ug of plasmid. The tested plasmid DNA encoded (1) BMPR1A-Fc, an inhibitor of bone morphogenetic protein (BMP) ligands; (2) TGFβRII-Fc, an inhibitor of transforming growth factor-beta ligands; and (3) insulin under the control of a constitutive promoter (CMV) (i.e., CMV-Bmpr1αfc, CMV-Tgfbr2fc, CMV-Ins). Notably, BMPR1A and TGFβRII are expressed as fusion proteins that comprise a C-terminal IgG Fc tag and an N-terminal secretion signal. Following electroporation, transfected adipocytes were cultured in vitro for 7 days and mRNA isolated. qPCR was performed to quantify expression of the respective transgenes (Bmpr1αfc, Tgfbr2fc, Ins). Our results demonstrate that adipocytes transfected with CMV-driven plasmids expressed the transgenes, while the negative control adipocytes (i.e., adipocytes electroporated without plasmid or untransfected adipocytes) did not express these transgenes (FIG. 5) (n>3 replicates).

### 3—Construction of Doxycycline-Inducible Constructs for Regulated Expression

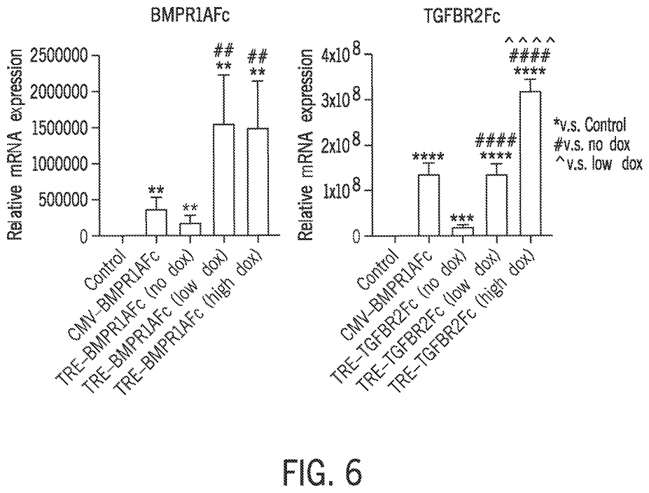

Additional doxycycline-inducible plasmids (TetOn-Bmpr1αfc and TetOn-Tgfbr2fc) have been constructed and preliminarily tested in non-adipocyte populations. The transfected HEK293 cells showed significant up-regulation of transgene expression upon treatment with doxycycline (1,000 ng/mL) (FIG. 6). We have also transfected human adipocytes with a doxycycline-inducible plasmid encoding BMPR1A-Fc (TetOn-Bmpr1αfc) and noted transgene expression.

### 4—Transfected Adipocytes Secrete Therapeutic Proteins Peptides

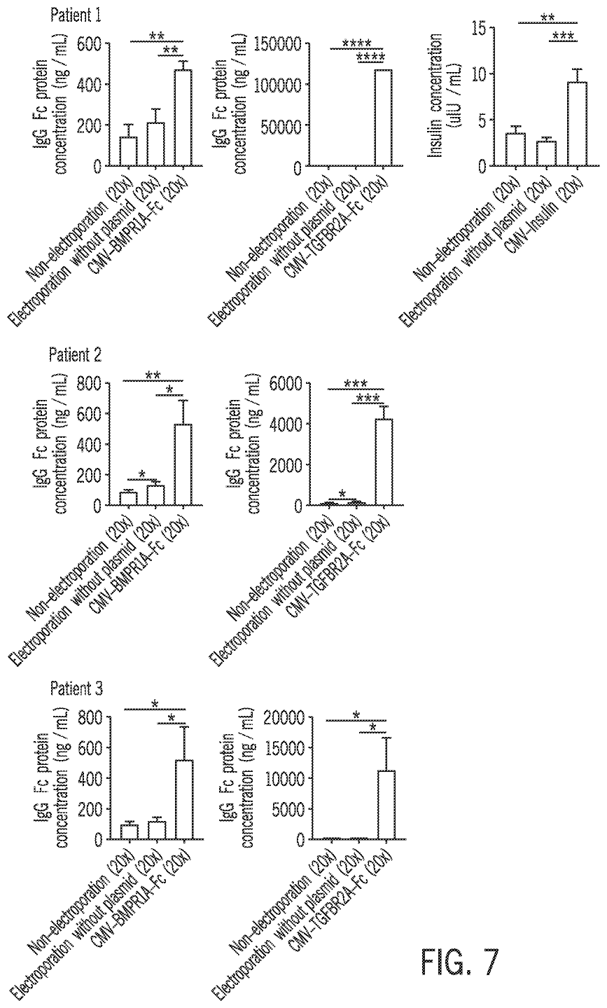

Although successful gene expression represents a significant advance in the use of adipocytes as a vehicle for cell therapy, demonstration of peptide secretion is critical for therapy delivery. Thus, we used ELISA to quantify TGFβRII-Fc, BMPR1A-Fc, or insulin protein in the conditioned media that had been used to culture transfected adipocytes. Controls were adipocytes that were not electroporated or adipocytes which were electroporated without plasmid. ELISA of media demonstrated significantly higher secretion of the desired BMPR1A-Fc and TGFβRII-Fc peptides by the transfected adipocytes as compared with negative controls (n>3 replicates) (FIG. 7).

### 5—Secreted BMPR1A-Fc and TGFβRII-Fc are Bioactive

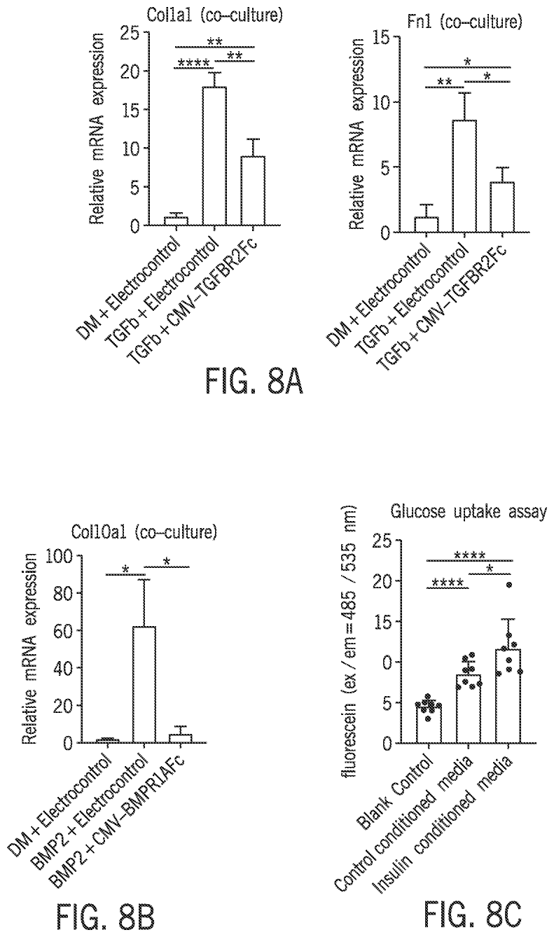

Secreted peptides may be rendered non-functional due to peptide misfolding. Therefore, we performed a series of experiments to confirm that the therapeutic agents secreted by adipocytes are bioactive. We confirmed bioactivity of secreted TGFβRII-Fc using a combination of conditioned media and co-culture experiments. Treatment with conditioned media from TGFβRII-Fc-expressing adipocytes or co-culture with TGFβRII-Fc-expressing adipocytes both reduced TGFβ-mediated transcriptional changes in TGFβ1-treated bone marrow-derived stromal cells (FIG. 8A) (n>3 replicates). Similarly, co-culture with BMPR1A-Fc-expressing adipocytes reduced BMP2-mediated transcriptional changes in BMP2-treated bone marrow-derived stromal cells (FIG. 8B) (n>3 replicates). Finally, a glucose uptake assay was performed to confirm the bioactivity of insulin present in the conditioned media of adipocytes transfected with the gene encoding insulin (Ins) (FIG. 8C). Overall, these findings provide evidence that the secreted agents possess bioactivity for use as therapeutic agents.

6—Electroporated Adipocytes Undergo Long-Term Engraftment into Recipient Sites

Red fluorescent mouse adipocytes derived from transgenic C57Bl/6 mice underwent the same transfection protocol used for human adipocytes (500V/5 msec/4 pulses) without delivery of plasmid to test survival and engraftment after exposure to electroporation conditions. Post-electroporation, RFP+ adipocytes were then delivered into the subcutaneous dorsum (100 uL), gastrocnemius muscle (50 uL), or Achilles tendon (10 uL) of same-strain mice. Mice were euthanized after 2 weeks and the sites were examined for presence of engrafted adipocytes. We found visible adipocyte clusters in the subcutaneous dorsum, confirmed with histologic evaluation for RFP+ adipocytes (FIG. 9A). Similarly, RFP+ adipocytes were identified in the gastrocnemius muscle and the Achilles tendon (FIG. 9B,C).

### 7—Post-Electroporation Cellular Protection Improves Expression

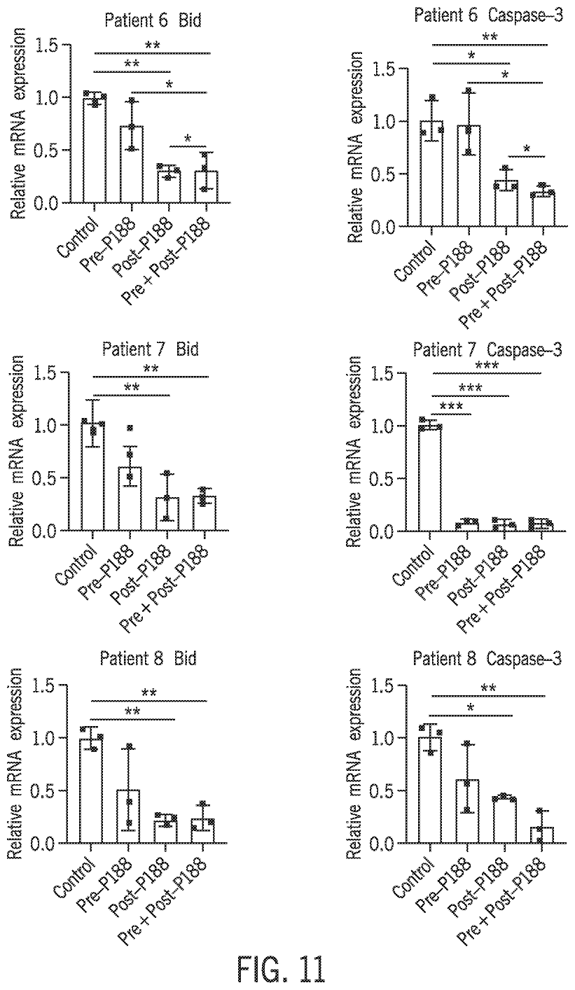

Adipocytes were treated with poloxamer-188, a polymeric compound that has been shown to stabilize cell membrane after injury, either (1) before, (2) before and after, or (3) after electroporation with the desired plasmid. Transgene expression levels were quantified. We noted increased expression levels with poloxamer-188 administration after electroporation, suggesting improved adipocyte stability after electroporation with poloxamer-188 treatment (FIG. 10). In addition, we performed a series of experiments to verify the effect of poloxamer-188 on adipocyte apoptosis after electroporation. In these experiments, treatment with poloxamer-188 resulted in downregulation of apoptosis-related genes, including Bid and Caspase-3 (FIG. 11).

### 8—Identification of Niche-Responsive Genes in Adipocytes

Human adipocytes were cultured in vitro with or without TGFβ1 (10 ng/mL) (n=6). qPCR revealed a significant increase in COL1A (181 v. 2.3, p<0.01) and CTGF transcripts (8.4 v. 1.0, p<0.01) relative to the house-keeping gene ACTB, supporting the use of promoters for these genes for closed-loop circuits.

### Example 2

Trauma-induced heterotopic ossification and traumatic muscle fibrosis are two related conditions which present substantial long-term morbidity to patients after injury. Although therapeutic options have been identified which can reduce or eliminate development of these post-traumatic sequelae, the absence of effective drug delivery strategies has served as a barrier to clinical translation. Biologic therapeutics, such as peptides or oligonucleotides, require in vivo synthesis, purification and formulation, storage, and exogenous delivery. While biomaterials and scaffolds have gained increasing attention as potential strategies for long-term release of embedded therapeutics, these strategies have their own shortcomings. For example, synthetic biomaterials may cause local tissue inflammation—the very same type of inflammation which we seek to mitigate in the setting of trauma-induced tissue pathology. Furthermore, these biomaterials and scaffolds may be difficult to disperse homogenously within the target tissues and have limited loading capacity. Therefore, alternative strategies for the delivery of peptide therapeutics are required.

In the following example, a description is provided of the use of an autologous adipose cell therapy to enable endogenous production and secretion of a therapeutic agent for the treatment of post-traumatic wound healing. It is envisioned that a patient's own tissues can be used to express and secrete a desired therapeutic biologic agent (i.e., a peptide or oligonucleotide), allowing for endogenous, bioresponsive dosing of the therapeutic agent.

Specifically, in this example, a description is provided of plans to express the therapeutic proteins BMPR1A-Fc and TGFβRII-Fc in mouse models of heterotopic ossification and muscle fibrosis, respectively. Additionally, a description is provided of plans to test various promoters for driving expression of these therapeutic proteins and to test the in vivo activity of the autologous adipocytes in pigs.

### 1—Establishing the Therapeutic Efficacy of Genetically Modified Autologous Adipocytes for the Treatment of Post-Traumatic Pathology

We hypothesize that adipocytes modified to express BMPR1A-Fc or TGFβRII-Fc will inhibit post-traumatic pathology in models of heterotopic ossification or muscle fibrosis.

C57Bl/6 mice that have undergone hindlimb tendon transection will serve as a model of heterotopic ossification. Control mice will receive no tendon injury at all. Mice will receive local administration of isogenic adipocytes isolated from C57Bl/6 mice and electroporated to deliver CMV-driven plasmids encoding BMPR1A-Fc or TGFβRII-Fc (i.e., CMV-Bmpr1αfc or CMV-Tgfbr2fc. Additional controls will include injured or uninjured mice which receive (1) no adipocyte injection, (2) injection of non-electroporated adipocytes, or (3) injection of adipocytes electroporated without plasmid delivery. Therefore, we will use a total of 10 groups of 10 mice, yielding a total of 100 mice. Evaluation of the mice will be performed with microCT scan to quantify the total heterotopic bone volume and histology to examine for location of the deposited adipocytes, and to confirm peptide production (i.e., using an anti-Fc antibody).

C57Bl/6 mice subjected to volumetric muscle loss (VML) injury to the gastrocnemius muscle will serve as a model of muscle fibrosis. The mice will be injected locally with isogenic adipocytes that have modified as described above. Similar controls will be used, such that a total of 10 treatment groups will be required with 10 mice/group. Histomorphometry will be performed to quantify fibrosis using picrosirius red staining (10 slides/mouse) using ImageJ. We will evaluate the engraftment and function of adipocytes using histologic staining.

### 2—Identifying Niche-Responsive Promoters for Autonomously Regulated Peptide Production

As is described above, we will use the CMV promoter to enable constitutive expression/production of the desired peptides. However, precise drug delivery requires a precise regulatory mechanism to modify expression levels. Thus, we will test the ability of various promoters to drive expression of BMPR1A-Fc or TGFβRII-Fc through closed-loop feedback. Plasmids which enable BMP2-responsive BMPR1A-Fc expression or TGFβ1-responsive TGFβRII-Fc expression will be synthesized.

We will first test plasmids that express BMPR1A-Fc or TGFβRII-Fc using the BMP-responsive element (BRE) or CAGA promoter, respectively (i.e., BRE-Bmpr1αfc or CAGA-Tgfbr2fc). Human adipocytes will undergo electroporation to deliver the specified promoter-driven plasmids; adipocytes will be treated with varying concentrations of BMP2 or TGFβ1, respectively (0-100 ng/mL); controls will receive 0 ng/mL of the corresponding ligand. We will quantify gene expression and peptide secretion using qPCR and ELISA at various time points after in vitro treatment, up to 2 weeks.

Next, we will identify additional candidate promoters that can be used to drive expression of the desired transgenes. We will treat adipocytes with BMP2 or TGFβ1, which are both recognized pathologic ligands in the processes of heterotopic ossification and muscle fibrosis, respectively. We will quantify the degree of transcriptional up-regulation for genes in the BMP or TGFβ pathways (BMP pathway: Col10, Alp, Runx2, Ocn, Dlx5 and TGFβ pathway: Col1, Fn, Ctgf) and identify promoters for these genes. We will then clone these promoters into our plasmids which already house the Bmpr1αfc or Tgfbr2fc gene.

Finally, we will transfect adipocytes with these synthesized plasmids and will quantify gene expression and peptide secretion using qPCR and ELISA after treatment with varying concentrations of BMP2 or TGFβ1 ligand.

### 3—Demonstration of In Vivo Bioactivity of Autologous Adipocytes in a Pig Model

Will we demonstrate in vivo activity in Yorkshire pigs, a large-animal model that approximates humans. Adipocytes will be isolated from Yorkshire pigs using liposuction and mechanical purification, similar to our approach with human adipocytes. Porcine adipocytes will be electroporated to deliver CMV-Bmpr1αfc or CMV-Tgfbr2fc. Transfected adipocytes will then be engrafted into the gastrocnemius muscle or hindlimb tendon of recipient pigs. Pigs will be euthanized after 4 weeks and the injection sites monitored for evidence of engraftment and peptide production using histologic evaluation and immunoblot of the local tissues. Systemic levels of the peptide will be monitored via serial serum ELISA for Fc peptides. Experiments will be performed with 5 recipient pigs for each delivery site, yielding 10 recipient pigs and 2 donor pigs at 15 weeks of age.

### Example 3

In the following example, a description is provided of engineered adipocytes that produce exosome-packaged therapeutic RNAs.

HEK293 cells and human adipocytes were cultured separately in media without fetal bovine serum. Exosomes were isolated and quantified using ExoQuick-TC (System Biosciences). Media from human control adipocytes and HEK293 cells had exosome concentrations of 4.2 ug/mL and 3.6 ug/mL respectively. After transfection with XMIR-LV-miR122, media from human adipocytes or HEK293 cells had exosome concentrations of 2.7 and 3.2 ug/mL respectively. These findings demonstrate that human adipocytes are capable of expressing and secreting exosomes, similar to HEK293 cells.

Recently, DNA constructs have been synthesized that direct the expression and packaging of therapeutic oligonucleotides, including microRNA (miRNA), into exosomes using validated nucleotide tags. We have acquired such plasmids from System Biosciences (XMIR Lentivector). Our plasmids are programmed to express miRNA-122.

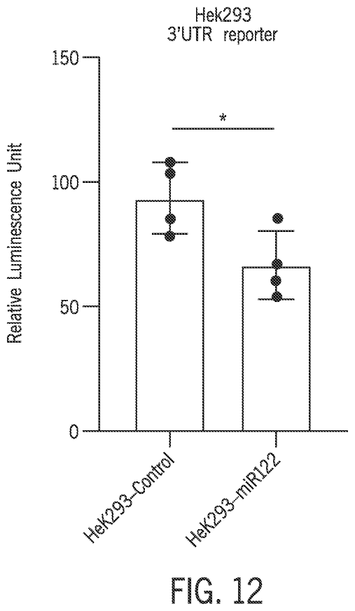

We first used these plasmids to test whether a luciferase assay system can be used to monitor exosome-packaged oligonucleotides. HEK293 cells were transfected with XMIR-LV-miR122 using Lipofectamine 3000. Separately, HEK293 cells were transfected with the miR-122 reporter plasmid, which down-regulates luciferase expression upon exposure to miR-122. As desired, we noted a significant reduction in luciferase activity in HEK293 reporter cells treated with conditioned media from HEK293/XMIR-LV-miR122 cells relative to conditioned media from HEK293/XMIR-null cells (FIG. 12).

Next, we confirmed that transfected adipocyte can also be used to express exosome-packaged miR-122. Briefly, human adipocytes or HEK293 cells (positive control) were transfected with null lentivector or XMIR-LV-miR122, and conditioned media was obtained after 3 days in culture. Media from both the human adipocytes and HEK293 cells transfected with the XMIR-LV-miR122 plasmid had exosome concentrations of 2.7 and 3.2 ug/mL respectively, demonstrating that human adipocytes secrete exosomes similar to HEK293 cells. In future work, luciferase assays will be performed to quantify the reduction in bioluminescence caused by miR-122 in human adipocytes, as described above.

### Example 4

In the following example, embodiments of various systems are disclosed for isolation of adipocytes. In general, each embodiment starts with fat from a subject. The fat is processed and using various methods to ultimately produces adipocytes that can be transfected and implanted in the subject.

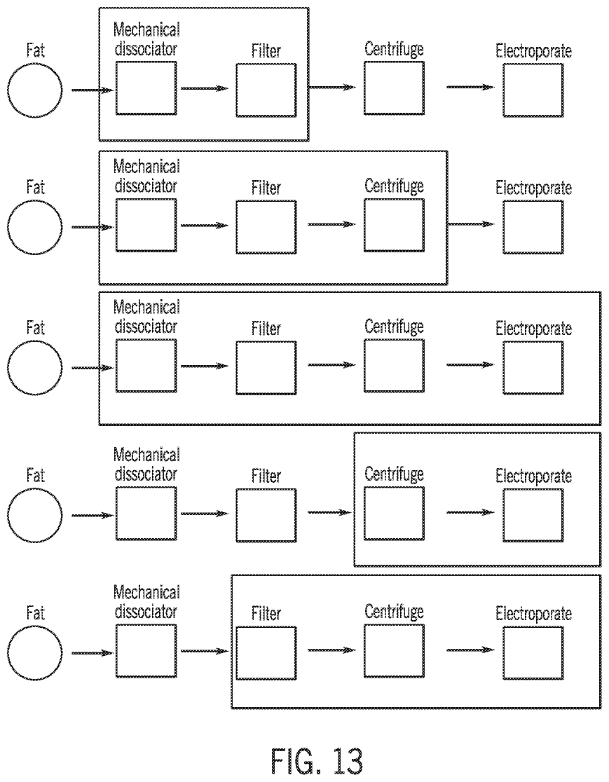

In each of the embodiments shown schematically in the diagram of FIG. 13, the system may include one or more of a mechanical dissociator, a filter, a centrifuge, or an electroporator. In each of these embodiments, certain components are surrounded by a rectangle, which indicates that those elements are coupled together as a single functional component in that particular embodiment. For example, the top row of FIG. 13 depicts an embodiment in which the mechanical dissociator and the filter are coupled together as a single functional component. The other four rows of FIG. 13 depict other possible combinations of elements, although many other combinations besides those shown in FIG. 13 are possible and are encompassed within the present disclosure.

What is claimed:

1. A method for treating a condition in a subject, the method comprising:
a) obtaining adipose tissue from the subject;
b) dissociating the adipose tissue into individual cells;
c) isolating adipocytes from the individual cells generated in step (b);
d) transfecting the isolated adipocytes with a polynucleotide comprising a promoter operably linked to a transgene encoding a therapeutic biologic agent (TBA); and
e) introducing the transfected adipocytes into the subject;
wherein the transfected adipocytes engraft into a target tissue and express and secrete the TBA, and wherein the TBA treats the condition.

2. The method of claim 1, wherein obtaining adipose tissue comprises obtaining a liquified liposuction aspirate.

3. The method of claim 1, wherein obtaining adipose tissue comprises obtaining whole adipose tissue, and wherein the method further comprises mechanically dissociating the adipose tissue into pieces prior to step (b).

4. The method of any one of the preceding claims, wherein isolating adipocytes further comprises:
i. filtering the individual cells generated in step (b) to obtain a filtrate;
ii. centrifuging the filtrate; and
iii. harvesting the adipocytes from the centrifuged filtrate.

5. The method of claim 4, wherein filtering the individual cells comprises filtering the individual cells through a filter with a pore size of 50-300 μm.

6. The method of any one of the preceding claims, wherein transfecting the isolated adipocytes comprises transfecting the isolated adipocytes using electroporation.

7. The method of any one of the preceding claims further comprising treating the adipocytes with a drug that improves cell survival.

8. The method of claim 7, wherein the drug is an anti-apoptotic agent or a membrane stabilizing agent.

9. The method of claim 8, wherein the drug is poloxamer-188.

10. The method of any one of the preceding claims, wherein the transfected adipocytes are introduced into the subject via subcutaneous injection, intraperitoneal injection, or injection into the target tissue.

11. The method of any one of the preceding claims, wherein the target tissue is a tissue selected from the group consisting of subcutaneous tissue, muscle, and tendon.

12. The method of any one of claims 1-11, wherein the condition is heterotopic ossification and the TBA is BMPR1A-Fc or TGFβRII-Fc.

13. The method of any one of claims 1-11, wherein the condition is muscle fibrosis and the TBA is TGFβRII-Fc.

14. The method of any one of claims 1-11, wherein the condition is type I diabetes and the TBA is insulin.

15. The method of any one of claims 1-11, wherein the condition is lymphedema and the TBA is VEGF-C or TGFβRII-Fc.

16. The method of any one of the preceding claims, wherein the transgene comprises a nucleotide sequence encoding at least one of a naturally-occurring gene, a synthetic gene, or a combination of a naturally-occurring gene and a synthetic gene.

17. A method of producing transfected adipocytes, the method comprising:
a) obtaining a plurality of isolated adipocytes; and
b) transfecting, using electroporation, the plurality of adipocytes with a polynucleotide comprising a promoter operably linked to a transgene.

18. The method of claim 17, wherein obtaining the plurality of adipocytes comprises dissociating adipose tissue into individual cells and isolating the dissociated adipocytes.

19. The method of claim 17 or 18, wherein electroporation is performed using the following electroporation settings: 500V, 4 pulses, and 5 msec per pulse.

20. The method of any one of claims 17-19 further comprising treating the transfected adipocytes with poloxamer-188 following electroporation.

21. The method of any one of claims 17-20, wherein the transgene encodes a TBA, and wherein the transfected adipocytes express and secrete the TBA.

22. The method of any one of the preceding claims, wherein the TBA comprises a secretion tag and/or a solubility tag.

23. The method of claim 22, wherein the TBA is BMPR1A-Fc or TGFβRII-Fc.

24. The method of claim 22, wherein the TBA is packaged into an exosome within the transfected adipocytes.

25. The method of any one of the preceding claims, wherein the polynucleotide is a plasmid or lentiviral vector.

26. The method of any one of the preceding claims, wherein the polynucleotide is a DNA donor template, and wherein the method further comprises transfecting the adipocytes with a gene editing enzyme and one or more guide nucleic acids.

27. The method of claim 26, wherein the DNA donor template comprises homology arms that target a safe harbor site within the adipocyte genome.

28. The method of claim 26 or 27, wherein the gene editing enzyme is Cas9.

29. The method of any one of the preceding claims, wherein the promoter is a constitutive promoter.

30. The method of claim 29, wherein the promoter is the cytomegalovirus (CMV) immediate early promoter.

31. The method of any one of claims 1-28, wherein the promoter is at least one of an inducible promoter or a niche-responsive promoter.

32. The method of claim 31, wherein the polynucleotide comprises a Tet-On system.

33. The method of any one of claims 17-32, wherein the transgene comprises a nucleotide sequence encoding at least one of a naturally-occurring gene, a synthetic gene, or a combination of a naturally-occurring gene and a synthetic gene.

34. An adipocyte produced according to the method of any of the preceding claims.

35. An isolated adipocyte transfected with a polynucleotide comprising a promoter operably linked to a transgene.

36. The isolated adipocyte of claim 35, wherein the transgene encodes a TBA, and wherein the adipocyte expresses and secretes the TBA.

37. The isolated adipocyte of claim 35, wherein the TBA comprises a secretion tag or a solubility tag.

38. The isolated adipocyte of claim 37, wherein the TBA is BMPR1A-Fc or TGFβRII-Fc.

39. The isolated adipocyte of claim 37, wherein the TBA is packaged into an exosome within the adipocyte.

40. The isolated adipocyte of any one of claims 35-39, wherein the polynucleotide is stably integrated into the genome of the adipocyte.

41. The isolated adipocyte of claim 40, wherein the polynucleotide is integrated into a safe harbor site within the adipocyte genome.

42. The isolated adipocyte of any one of claims 35-41, wherein the promoter is a constitutive promoter.

43. The isolated adipocyte of claim 42, wherein the promoter is the cytomegalovirus (CMV) immediate early promoter.

44. The isolated adipocyte of any one of claims 35-41, wherein the promoter is an inducible promoter.

45. The isolated adipocyte of claim 44, wherein the polynucleotide comprises a Tet-On system.

46. The isolated adipocyte of any one of claims 35-45, wherein the adipocyte is a mature, primary adipocyte.

47. The isolated adipocyte of any one of claims 17-32, wherein the transgene comprises a nucleotide sequence encoding at least one of a naturally-occurring gene, a synthetic gene, or a combination of a naturally-occurring gene and a synthetic gene.


SEQUENCE LISTING (9 sequences)

SEQ ID NO: 1 - protein, 389 aa, Artificial Sequence
  Synthetic - BMPR1A-Fc
  MISC_FEATURE (1)..(23): signal peptide of BMPR1A
  MISC_FEATURE (24)..(152): functional domain of BMPR1A
  misc_feature (153)..(158): linker
  misc_feature (159)..(389): Fc tag
MPQLYIYIRLLGAYLFIISRVGGQNLDSMLHGTGMKSDSDQKKSENGVTLAPEDTLPFLKCYCSGHCPDDAINNTCITNGHCFAIIEEDDQGETTLASGCMKYEGSDFQCKDSPKAQLRRTIECCRTNLCNQYLQPTLPPVVIGPFFDGSIRIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - DNA, 1167 bp, Artificial Sequence
  Synthetic - BMPR1A-Fc
  misc_feature (1)..(69): signal peptide of BMPR1A
  misc_feature (70)..(456): functional domain of BMPR1A
  misc_feature (457)..(474): linker
  misc_feature (475)..(1167): Fc tag
atgccgcagctgtatatttatattcgcctgctgggcgcgtatctgtttattattagccgcgtgggcggccagaacctggatagcatgctgcatggcaccggcatgaaaagcgatagcgatcagaaaaaaagcgaaaacggcgtgaccctggcgccggaagataccctgccgtttctgaaatgctattgcagcggccattgcccggatgatgcgattaacaacacctgcattaccaacggccattgctttgcgattattgaagaagatgatcagggcgaaaccaccctggcgagcggctgcatgaaatatgaaggcagcgattttcagtgcaaagatagcccgaaagcgcagctgcgccgcaccattgaatgctgccgcaccaacctgtgcaaccagtatctgcagccgaccctgccgccggtggtgattggcccgttttttgatggcagcattcgcattgaaggccgcatggatccgaaaagctgcgataaaacccatacctgcccgccgtgcccggcgccggaactgctgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagcaaactgaccgtggataaaagccgctggcagcagggcaacgtgtttagctgcagcgtgatgcatgaagcgctgcataaccattatacccagaaaagcctgagcctgagcccgggcaaa

SEQ ID NO: 3 - protein, 392 aa, Artificial Sequence
  Synthetic - TGFBII-Fc
  MISC_FEATURE (1)..(22): signal peptide of TGFBII
  MISC_FEATURE (23)..(159): functional domain of TGFBII
  MISC_FEATURE (160)..(161): linker
  MISC_FEATURE (162)..(392): Fc tag
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - DNA, 1176 bp, Artificial Sequence
  Synthetic - TGFBII-Fc
  misc_feature (1)..(66): signal peptide of TGFBII
  misc_feature (67)..(477): functional domain of TGFBII
  misc_feature (478)..(483): linker
  misc_feature (484)..(1176): Fc tag
atgggccgcggcctgctgcgcggcctgtggccgctgcatattgtgctgtggacccgcattgcgagcaccattccgccgcatgtgcagaaaagcgtgaacaacgatatgattgtgaccgataacaacggcgcggtgaaatttccgcagctgtgcaaattttgcgatgtgcgctttagcacctgcgataaccagaaaagctgcatgagcaactgcagcattaccagcatttgcgaaaaaccgcaggaagtgtgcgtggcggtgtggcgcaaaaacgatgaaaacattaccctggaaaccgtgtgccatgatccgaaactgccgtatcatgattttattctggaagatgcggcgagcccgaaatgcattatgaaagaaaaaaaaaaaccgggcgaaaccttttttatgtgcagctgcagcagcgatgaatgcaacgataacattatttttagcgaagaatataacaccagcaacccggatatggatccgaaaagctgcgataaaacccatacctgcccgccgtgcccggcgccggaactgctgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagcaaactgaccgtggataaaagccgctggcagcagggcaacgtgtttagctgcagcgtgatgcatgaagcgctgcataaccattatacccagaaaagcctgagcctgagcccgggcaaa

SEQ ID NO: 5 - protein, 532 aa, Homo sapiens
  misc_feature (1)..(532): bone morphogenetic protein receptor type-1A (BMPR1A) precursor
  misc_feature (1)..(23): signal peptide
  misc_feature (24)..(152): functional domain
MPQLYIYIRLLGAYLFIISRVQGQNLDSMLHGTGMKSDSDQKKSENGVTLAPEDTLPFLKCYCSGHCPDDAINNTCITNGHCFAIIEEDDQGETTLASGCMKYEGSDFQCKDSPKAQLRRTIECCRTNLCNQYLQPTLPPVVIGPFFDGSIRWLVLLISMAVCIIAMIIFSSCFCYKHYCKSISSRRRYNRDLEQDEAFIPVGESLKDLIDQSQSSGSGSGLPLLVQRTIAKQIQMVRQVGKGRYGEVWMGKWRGEKVAVKVFFTTEEASWFRETEIYQTVLMRHENILGFIAADIKGTGSWTQLYLITDYHENGSLYDFLKCATLDTRALLKLAYSAACGLCHLHTEIYGTQGKPAIAHRDLKSKNILIKKNGSCCIADLGLAVKFNSDTNEVDVPLNTRVGTKRYMAPEVLDESLNKNHFQPYIMADIYSFGLIIWEMARRCITGGIVEEYQLPYYNMVPSDPSYEDMREVVCVKRLRPIVSNRWNSDECLRAVLKLMSECWAHNPASRLTALRIKKTLAKMVESQDVKI

SEQ ID NO: 6 - protein, 567 aa, Homo sapiens
  MISC_FEATURE (1)..(567): TGF-beta receptor type-2 isoform B (TGFBII) precursor
  MISC_FEATURE (1)..(22): signal peptide
  MISC_FEATURE (23)..(159): functional domain
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 7 - protein, 6 aa, Artificial Sequence
  Synthetic - linker peptide used in BMPR1A-Fc
IEGRMD

SEQ ID NO: 8 - protein, 330 aa, Homo sapiens [sequence omitted]

SEQ ID NO: 9 - protein, 231 aa, Artificial Sequence
  Synthetic - Fc tag (amino acids 100-330 of SEQ ID NO8)
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK